(19) United States
(12) Patent Application Publication
(10) Pub. No.: US 2022/0233492 A1
(43) Pub. Date: Jul. 28, 2022

(54) METHOD OF PREVENTING OR TREATING POSTOPERATIVE PAIN

(71) Applicant: Vivozon Inc., Yongin-si, Gyeonggi-do (KR)

(72) Inventors: Doo Hyun LEE, Lawrence, NJ (US); Sunyoung CHO, Princeton, NJ (US)

(73) Assignee: Vivozon Inc., Yongin-si, Gyeonggi-do (KR)

(21) Appl. No.: 17/596,397

(22) PCT Filed: Jun. 19, 2020
(86) PCT No.: PCT/KR2020/007946

§ 371 Date: Dec. 9, 2021

Related U.S. Application Data
(60) Provisional application No. 62/864,134, filed on Jun. 20, 2019.
(60) Provisional application No. 62/951,088, filed on Dec. 20, 2019.

(51) Int. Cl. A61K 31/351; A61P 29/00

(52) U.S. Cl. CPC A61K 31/351; A61P 29/00

(57) ABSTRACT

A method for treating or preempting pain including postoperative pain following a soft or hard tissue surgery is disclosed. Also disclosed is a method for reducing an postoperative analgesic need, including administering a compound of Formula (I) or a salt thereof.

### CROSS REFERENCE TO RELATED APPLICATIONS

This application is a National Stage Entry Application of PCT/KR2020/007946 filed Jun. 19, 2020, which claims priority to U.S. Provisional Application No. 62/864,134 filed on Jun. 20, 2019 and U.S. Provisional Application No. 62/951,088 filed on Dec. 20, 2019. The entire disclosure of the applications identified in this paragraph is incorporated herein by reference.

### TECHNICAL FIELD

The disclosure relates to, among other things, methods for preventing and/or treating pain including postoperative pain.

### BACKGROUND ART

An appropriate and adequate management of perioperative pain during postoperative care is critical for both treating acute pain and preventing postsurgical chronic pain caused by either ongoing inflammation or a manifestation of neuropathic pain. Apfelbaum J L, et al., Postoperative pain experience: results from a national survey suggest postoperative pain continues to be undermanaged. Anesth Analg 2003; 97:534-540.

Alleviating pain and suffering in postoperative patients is an area of special focus in clinical medicine, especially with the growing number of out-patient operations performed each year. The most widely used agents, cyclooxygenase inhibitors (e.g., ibuprofen) and opioids (e.g., morphine, fentanyl), have significant side effects including gastrointestinal irritation/bleeding and respiratory depression. The high incidence of nausea and vomiting related to opioids is especially problematic in the postoperative period. Therapeutic agents aimed at treating postoperative pain while avoiding detrimental side effects are not easily developed because the molecular targets for these agents are distributed widely throughout the body and mediate diverse physiological actions. Despite the significant clinical need to inhibit pain and inflammation, as well as vasospasm, smooth muscle spasm and restenosis, methods for the delivery of inhibitors of pain, inflammation, spasm and restenosis at effective dosages while minimizing adverse systemic side effects have not been developed. As an example, conventional (i.e., intravenous, oral, subcutaneous or intramuscular) methods of administration of opiates in therapeutic doses frequently is associated with significant adverse side effects, including severe respiratory depression, changes in mood, mental clouding, profound nausea and vomiting.

Postoperative or post-operation pain (interchangeably termed, post-surgical pain or post-incisional pain) is often considered a variety of inflammatory pain. While there may be an “inflammatory” component to post-operation pain, clearly additional mechanisms are involved. For example, during operation or other injury, both vasculature and nerves are cut or torn.

Different mechanisms involved in surgical or injury-induced pain as compared to inflammation is exemplified by the varying pharmacology and underlying anatomical substrates of pain relief in the two conditions. Yamamoto, et al., (Brian Res. 909(1-2):138-144 (2001)) have shown that inhibition of spinal N-acetyl-alpha-linked acidic dipeptidase (NAALADase) causes a marked attenuation of mechanical pain which accompanies the inflammatory stimulus of carrageenan injection. However, in parallel experiments where NAALADase was inhibited in an identical fashion after an incision, there was no attenuation of mechanical pain. These observations demonstrate that the biochemistry or pharmacology underlying post-operation pain is distinct from those underlying inflammatory pain. In addition to the obvious differences in post-operative or injury-induced pain from inflammatory, visceral or neuropathic pain, it has been speculated that the mechanisms involved in postoperative pain (or injury-induced pain) are clearly different from other pains. Further, the utility of a particular pharmacological (or other) intervention in treating postoperative pain is not predictable by testing that pharmacological agent or intervention in inflammatory, visceral or neuropathic pain models.

High levels of postoperative pain have been associated with an increased risk of chronic pain as a consequence of surgery. Tasmuth T et al., Acta Oncol 1997; 36:625-63010,16, 18-20. Therefore, an aggressive treatment of postoperative pain may be important in chronic-pain patients because they may represent a population that is particularly vulnerable to complications or are at risk for chronic postsurgical pain. Carroll et al., Regional Anesthesia and Pain Medicine, Vol. 29 No. 6 (2004), pp. 576-591.

Various factors, for example demographic factors such as gender and age; psychologic conditions such as depression, anxiety, and neuroticism; preexisting pain conditions; and the preoperative use of opioids can be used to predict poorer pain control and increased analgesic requirements in the postoperative period. For example, chronically opioid consumed patients experience increased and prolonged postoperative pain, despite an increased postoperative opioid consumption. Carroll et al., Regional Anesthesia and Pain Medicine, Vol. 29 No. 6 (2004), pp. 576-591.

A postoperative pain management generally has two goals: first goal is to provide a quick onset of analgesic or pain relief and second goal of reducing or modulating the duration, quality, and intensity of pain that a patient experiences in the post-surgical period. Despite advances in pain management in recent decades, approximately 60% of surgical patients experience moderate, severe, or extreme pain postoperatively. Chou R, et al. Management of Postoperative Pain: A Clinical Practice Guideline From the American Pain Society, the American Society of Regional Anesthesia and Pain Medicine, and the American Society of Anesthesiologists' Committee on Regional Anesthesia, Executive Committee, and Administrative Council. J Pain 17:131-157, 2016. Therefore, there still is a need for improved methods for treating post-surgical acute pain.

U.S. Pat. No. 9,359,346 describes novel benzamide derivatives including opiranserin and pharmaceutically acceptable salts thereof and their use for the treatment of pain or itching. The entire content of U.S. Pat. No. 9,359,346 is incorporated herein by reference.

### DISCLOSURE

### Technical Problem

The present disclosure is directed to a method of treating pain in a subject.

An aspect of the present disclosure provides a method of providing a safe and effective alternative or adjuvant injectable analgesic for use to treat acute pain in postoperative setting.

In an aspect, the present disclosure provides a method of treating acute postoperative pain of a subject.

Another aspect of the present disclosure is directed to a method for treating pain in, for example the acute postoperative setting which is or may be opioid-sparing.

### Solution to Problem

The present disclosure provides a method of treating or preventing pain including postoperative pain in a subject in need thereof, comprising administration of a compound of Formula (I) or a pharmaceutically acceptable salt thereof:

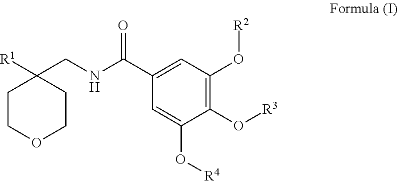

wherein R¹ is —NR⁵R⁶ or morpholinyl; R², R³, and R⁴ are each independently selected from the group consisting of alkyl, aryl, and arylalkyl; and R⁵ and R⁶ are each independently selected from the group consisting of —H, alkyl, and arylalkyl.

According to one aspect, there is provided a method of treatment of pain in a subject, comprising: administering to the subject a pharmaceutically effective amount of the compound of Formula (I) or pharmaceutically acceptable salt thereof, wherein the compound is administered by continuous intravenous (I.V.) infusion to the subject, and wherein the pain is moderate to severe pain. In an embodiment, the pain is acute postoperative pain.

In an aspect of the present disclosure, the compound of Formula (I) is (4-butoxy-N-((4-(dimethylamino)tetrahydro-2H-pyran-4-yl)methyl)-3,5-dimethoxybenzamide) represented by the following formula:

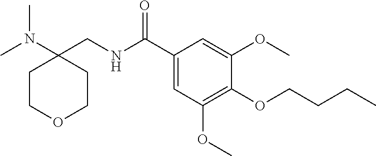 [SMILES: CCCCOc1c(OC)cc(C(=O)NCC2(N(C)C)CCOCC2)cc1OC]

or a pharmaceutically acceptable salt thereof. In an exemplary embodiment, the compound is (4-butoxy-N-((4-(dimethylamino)tetrahydro-2H-pyran-4-yl)methyl)-3,5-dimethoxybenzamide) hydrochloride.

According to another aspect, there is provided a method for reducing an amount of an postoperative analgesia administered for pain treatment of a subject, comprising administering to the subject an effective amount of a compound of Formula (I) or pharmaceutically acceptable salt thereof, wherein the compound is administered by continuous I.V. infusion to the subject, and wherein the pain is moderate to severe pain.

According to yet another aspect, there is provided a method for reducing a need of analgesia by a subject suffering from postoperative pain, comprising administering to the subject a pharmaceutically effective amount of the compound of Formula (I) or pharmaceutically acceptable salt thereof, wherein the compound is administered by continuous I.V. infusion to the subject, and wherein the pain is moderate to severe pain.

According to yet another aspect, there is provided a pharmaceutical composition for reducing a need of a postoperative analgesia by a subject underwent operation, comprising an effective amount of a compound of Formula (I) or pharmaceutically acceptable salt thereof, together with a pharmaceutically acceptable carrier.

According to yet another aspect, there is provided a pharmaceutical composition for treating postoperative pain of a subject underwent operation, comprising an effective amount of a compound of Formula (I) or pharmaceutically acceptable salt thereof, together with a pharmaceutically acceptable carrier.

### BRIEF DESCRIPTION OF DRAWINGS

FIG. 1 shows pain intensity in VVZ-149 and placebo groups, as explained in Example 1.

FIG. 2 shows a total amount of opioid consumption (hydromorphone, mg) per 2 hours, as explained in Example 1. The total opioid consumption is the summated amount of hydromorphone administered via patient-controlled analgesia (PCA) and rescue dosing requested by patients (VVZ-149 group: 37 patients, placebo group: 16 patients). Data are represented as means±SEMs; * p<0.05.

FIG. 3 shows numbers of PCA demands per 2 hours, as explained in Example 1. (WZ-149 group: 37 patients, placebo group: 16 patients). Data are represented as means±SEMs; * p<0.05, ** p<0.01, ***p<0.001.

FIG. 4 shows the total number of PCA demands from 0 to 12 hours post-emergence, as explained in Example 1 (WZ-149 group: 37 patients, placebo group: 16 patients). Data are represented as means±SEMs; * p<0.05. As a patient's self administered opioid through a PCA machine, hydromorphine was used. The results show that the number of patient's PCA demands in the VVZ-149 group is significantly lower during the post-emergence 24 hour period than that of the placebo group. The term “the number of PCD demands” used herein is a number of button press by the patient on the PCA machine.

FIG. 5a and FIG. 5b show pain intensity (FIG. 5a) and total opioid consumption (hydromorphone, mg) per 2 hours (FIG. 5b) in a subgroup of patients who reported a predose PI over 7, as explained in Example 1. The subgroup is composed of a total of 29 patients, 20 patients in the WZ-149 group and 9 patients in the placebo group. Data are represented as means±SEMs; * p<0.05, ** p<0.01.

FIG. 6 shows three-dimensional mesh plots of relationship between anxiety (HADS-A), depression (HADS-D), and pain catastrophizing (PCS) in the non-rescued, and rescued patients, as explained in Example 1 (non-rescued 25 patients, rescued 27 patients).

FIG. 7 shows pain intensity and the total opioid consumption (hydromorphone, mg) of the rescued group (VVZ-149 group 19 patients, placebo group 8 patients) (FIG. 7b) as explained in Example 1. Data are represented as means±SEMs. * p<0.05, ** p<0.01.

FIG. 8 shows the total number of opioid requests in VVZ-149 group and placebo group during 0-24 hours, as explained in Example 2 (WZ-149 group 149 patients, placebo group 152 patients). Data are represented as means±SEMs; * p<0.05.

FIG. 9 shows the total amount of opioid consumed postoperatively during 0-6 hours, 0-12 hours, and 0-24 hours, as explained in Example 2. (WZ-149 group 149 patients, placebo group 152 patients). Data are represented as means±SEMs; * p<0.05, ** p<0.01, *** p<0.001

FIG. 10 shows the proportion of opioid-free subjects during each of 0-6, 6-12, 12-18 and 18-24 hour interval, as explained in Example 2 (VVZ-149 group 152 patients, placebo group 154 patients). Data are represented as means±SEMs; * p<0.05, *** p<0.001.

FIG. 11 shows the amount of opioid consumed intraoperatively during surgery, as explained in Example 2. (WZ-149 group 152 patients, placebo group 154 patients). Data are represented as means±SEMs.

FIG. 12 shows the pain intensity in VVZ-149 and placebo groups, as explained in Example 4 (VVZ-149 group 30 patients, placebo group 29 patients). Data are represented as means±SEMs; * p<0.05.

FIG. 13 shows the total number of PCA demands per 2 hours throughout 24 hours as explained in Example 4 (VVZ-149 group 30 patients, placebo group 29 patients). Data are represented as means±SEMs; * p<0.05

FIG. 14 shows the total opioid consumption (fentanyl equivalent, μg) for 10 hours post-emergence, as explained in Example 4 (WZ-149 group 30 patients, placebo group 29 patients). Data are represented as means±SEMs; ** p<0.01

FIG. 15a and FIG. 15b show the pain intensity and total opioid consumption (hydromorphone, mg) of the rescued group (VVZ-149 group 17 patients, placebo group 19 patients) (FIG. 15b) and non-rescued group (VVZ-149 group 13 patients, placebo group 10 patients) (FIG. 15a), as explained in Example 4. Data are represented as means±SEMs. * p<0.05, ** p<0.01

FIG. 16a shows the pain intensity in VVZ-149 and placebo groups (VVZ-149 group 30 patients, placebo group 30 patients), and FIG. 16b shows the pain intensity of subgroup with predose PI≥7 (FIG. 16b) (VVZ-149 group 17 patients, placebo group 15 patients). FIG. 16c shows the pain intensity of subgroup with predose PI≥6 (VVZ-149 group 22 patients, placebo group 20 patients), and FIG. 16d shows the pain intensity of subgroup with predose PI≥5 (VVZ-149 group 27 patients, placebo group 25 patients), as explained in Example 3. Data are represented as means±SEMs; * p<0.05, ** p<0.01.

FIGS. 17a-17b show the Area Under the Curve (AUC) of pain intensity and total opioid consumption (morphine, mg) in a subgroup with a predose PI≥7 during 0-12 and 0-24 hours, as explained in Example 3 (VVZ-149 group 18 patients, placebo group 15 patients). Data are represented as means±SEMs

FIG. 18 shows the Kaplan-Meier graph of the percentage of subjects who reported perceptible pain relief in relation to time, as explained in Example 3. *p<0.05

### MODES

The methods of the embodiments are described in further detail in the following sections.

### Definitions

The term “acute pain” as used herein means pain that has a sudden onset and commonly declines over a short time (days, hours, minutes) and follows injury to the body and which generally disappears when the bodily injury heals.

The term “effective analgesia” or “analgesia” as used herein is defined as a satisfactory reduction in or elimination of pain, along with the process of a tolerable level of side effects, as determined by the human patient.

The term “postoperative analgesia” as used herein as defined as an analgesia administered to manage postoperative pain. A kind and amount of postoperative analgesia can be determined by a skilled artisan. Examples of postoperative analgesia may include, but is not limited to, an NSAID, an opioid, or a combination thereof. As an opioid, morphine, codeine, fentanyl transdermal, hydrocodone hydromorphone, oxycodone, oxymorphone, tapentadol, or tramadol can be exemplified.

The term “effective pain management” as used herein means as the objective evaluation of a human patient's response (pain expressed versus side effects) to analgesic treatment by a physician as well as subjective evaluation of therapeutic treatment by the patient undergoing such treatment. The skilled artisan will understand that effective pain management will vary according to many factors, including interindividual variability in patients.

The term “breakthrough pain” means pain which the patient experiences despite the fact that the patient is being administered generally effective amounts of, e.g., an analgesic.

The term “rescue” as used herein refers to a dose of an analgesic which is administered to a patient experiencing breakthrough pain.

An “effective dose” or “effective amount” is an amount sufficient to effect beneficial or desired clinical results including alleviation or reduction in pain. For purposes of subject embodiments, an effective amount of a compound of Formula (I) is an amount sufficient to treat, ameliorate, reduce the intensity of or prevent post-operative pain. In some embodiments, the “effective amount” may reduce the pain of ongoing pain and/or breakthrough pain (including ambulatory pain and touch-evoked pain).

The term “parenterally” as used herein includes subcutaneous injection, intravenous injection, intramuscular injection, intradermal injection, or infusion techniques.

The term “patient” or “subject” as used herein refers to a warm blooded animal such as a mammal which is the subject of trauma, e.g., surgical trauma. It is understood that at least humans, dogs, cats, and mice are within the scope of the meaning of the term.

The term “treat” or “treatment,” or a derivative thereof, as used herein, contemplates partial or complete inhibition of pain or alleviating the degree of pain or reducing the duration of pain, when a composition of the embodiment is administered before, during, or after a surgical operation.

As used herein, the term “perioperative” encompasses application intraprocedurally, pre- and intraprocedurally, intra- and postprocedurally, and pre-, intra- and postprocedurally.

As used herein, the term “about” and “approximately,” which are used interchangeably, means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” or “approximately” in the context of the mass of an ion or the mass/charge ratio of an ion refers to +/−0.50 atomic mass unit. Furthermore, for instance, the term “about” or “approximately” in the context of a duration or a lapse of a period of time refers to +/−1%, 2%, 3%, 4%, 5%, 6%, 7%, 8%, 9%, 10%, 11%, 12%, 13%, 14%, or 15% of the indicated duration or the period.

As used herein, ranges and amounts can be expressed as “about” or “approximately” a particular value or range. About also includes the exact amount. Thus, “about 1000 mg” means “about 1000 mg” and also “1000 mg.” Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment also carries with it the alternative meaning of “may not” or “cannot.” As such, if the present specification discloses that an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar referents used in the context of describing the present embodiments are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present embodiments and does not pose a limitation on the scope of the embodiments otherwise claimed. No language in the present specification should be construed as indicating any non-claimed element essential to the practice of the embodiments.

Compounds

Compounds useful for treating pain such as postoperative and/or reducing an amount of an analgesic to treat postoperative pain and/or reducing a need of an analgesic to treat postoperative pain, and methods for synthesizing these compounds were described in U.S. Pat. No. 9,359,346, content of which is incorporated herein by reference:

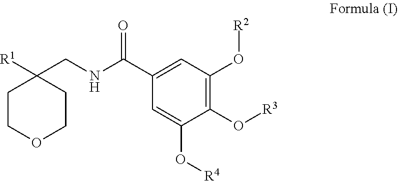

wherein R¹ is —NR⁵R⁶ or morpholinyl; R², R³, and R⁴ are each independently selected from the group consisting of alkyl, aryl, and arylalkyl; and R⁵ and R⁶ are each independently selected from the group consisting of —H, alkyl, and arylalkyl.

Non-limiting examples of Formula (I) compound include:

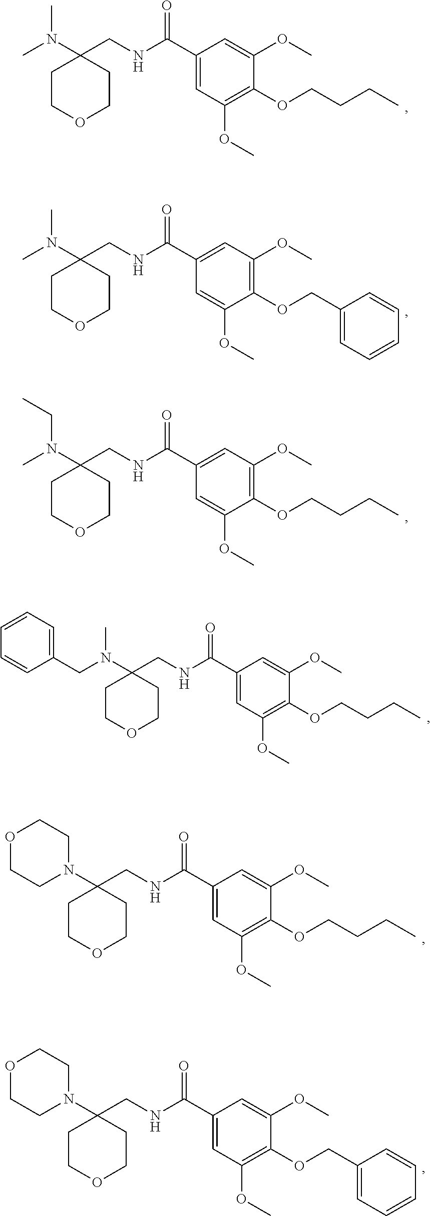

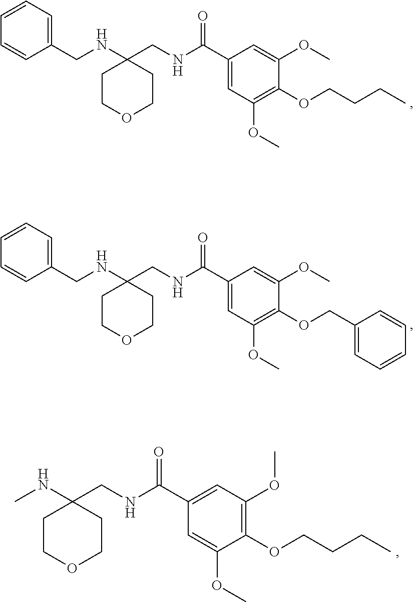

and a pharmaceutically acceptable salt thereof.

VVZ-149 (4-butoxy-N-((4-(dimethylamino)tetrahydro-2H-pyran-4-yl) methyl)-3,5-dimethoxybenzamide) is a compound in benzamide family, and represented by the following chemical formula:

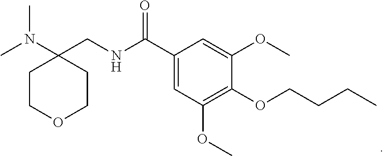 [SMILES: CCCCOc1c(OC)cc(C(=O)NCC2(N(C)C)CCOCC2)cc1OC]

VVZ-149 is also known as opiranserin. As used herein, VVZ-149 represents 4-butoxy-N-((4-(dimethylamino)tetrahydro-2H-pyran-4-yl)methyl)-3,5-dimethoxy benzamide (MW 394.51) as well as a pharmaceutically acceptable salt thereof, unless specified otherwise. As used herein, the term VVZ-149 or opiranserin also refers to 4-butoxy-N-((4-(dimethylamino)tetrahydro-2H-pyran-4-yl)methyl)-3,5-dimethoxy benzamide or a pharmaceutically acceptable salt thereof, unless specified otherwise.

In one embodiment, VVZ-149 (opiranserin) is 4-butoxy-N-((4-(dimethylamino)tetrahydro-2H-pyran-4-yl)methyl)-3,5-dimethoxybenzamide hydrochloride (MW 430.97) of the following structure:

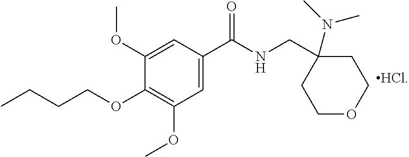 [SMILES: CCCCOc1c(OC)cc(C(=O)NCC2(N(C)C)CCOCC2)cc1OC]

VVZ-149 is a dual inhibitor/antagonist of type 2 glycine transporter (GlyT2) and type 2A serotonin receptor (5HT2A)m, and metabolized into N-desmethyl 4-butoxy-N-((4-(dimethylamino)tetrahydro-2H-pyran-4-yl)methyl)-3,5-dimethoxybenzamide (referred to herein as VVZ-368).

In double-blinded, randomized preclinical studies with pharmacokinetic/pharmacodynamic (PK/PD) correlation analyses, VVZ-149 has demonstrated dose-dependent pain relief and an anti-allodynic effect comparable to morphine or gabapentin without significant sedative or adverse effects. No clinically significant adverse events (AEs) occurred during a study on healthy human subjects or subjects undergoing major surgeries.

### Formulation

The injection formulation may be provided in a form of solution of 10 mg hydrochloride form/mL water for injection (WFI), which can be mixed with saline for infusion. For example, 100 mL of injection (i.e., 1,000 mg of opiranserin as a hydrochloride form) can be mixed with 400 mL of saline to administer a 1,000 mg dosage to a subject.

The formulation can be lyophilized, which can be reconstructed for use.

The intravenous formulation in accordance with the embodiment typically includes a compound of Formula (I) in the form of its hydrochloride salt (e.g., VVZ-149 HCl, MW 430.97). However, one of ordinary skill in the art will appreciate that other forms of opiranserin may be used, including, but not limited to, pharmaceutically acceptable salts of opiranserin and adjust the accurate dose accordingly. Such pharmaceutically acceptable salts may include, but are not limited to, metal salts such as sodium salt, potassium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

Treatment of Moderate to Severe Pain

One aspect of the present invention is directed to a method of treatment of pain in a subject, comprising administering to the subject an effective amount of a compound of Formula (I) or a pharmaceutically acceptable salt thereof, wherein the compound is administered by continuous I.V. infusion to the subject, and wherein the pain is moderate to severe pain.

One aspect of the present invention is directed in part to a compound of Formula (I) in a pharmaceutically acceptable sterile solution formulation containing an effective dose of the compound, and a method of administration of the same for the treatment of pain, e.g., perioperatively such as prior to the operation, during the operation, and/or after the operation. An injection of a compound of Formula (I) in accordance with the present embodiment will fulfill an important need by providing a safe and effective alternative injectable analgesic for use in effective pain management of acute postoperative pain.

One aspect of the present invention is directed to a method for reducing an amount of an analgesia administered for pain treatment in a subject who underwent an operation and is under postoperative pain treatment, the method comprising administering to the subject an effective amount of a compound of Formula (I) or a pharmaceutically acceptable salt thereof. In an embodiment, the administration of the compound of Formula (I) or a salt thereof is carried out prior to the operation (“preoperative dosing” or “preoperative administration”). In another embodiment, the administration of the compound of Formula (I) or a salt thereof commences prior to the operation (for example, about 1 hr—about 30 minutes prior to anesthesia induction) and continues during the operation and ends during the operation or after the operation) (“pre-, infra- and post-operation dosing” or “pre- and intra-operation dosing”). In another embodiment, the administration of the compound of Formula (I) or a salt thereof carries out during the operation (“intra-operation dosing”). In yet another embodiment, the administration of the compound of Formula (I) or a salt thereof commences during the operation or before emergence from anesthesia and continues after the operation (“intra- and post-operation dosing”). In still another embodiment, the administration of the compound of Formula (I) or a salt thereof commences prior is carried out after emergence from the operation.

Conventional postoperative pain treatment includes an administration of an analgesic after the completion of the operation. The method according to the embodiment is characterized that the treatment starts before starting an operation, e.g., about 30 minutes earlier than induction of anesthesia.

The administration may be carried out continuously by IV infusion for about 4 hours to about 15 hours. In another embodiment, the IV infusion could be carried out for about 5 hours to about 15 hours. In another embodiment, the IV infusion could be carried out for about 6 to about 15 hours, about 7 to about 15 hours, about 8 to about 15 hours, about 9 to about 15 hours, about 10 to about 15 hours, about 4 to about 12 hours, about 5 to about 12 hours, about 6 to about 12 hours, about 7 to about 12 hours, about 8 to about 12 hours, about 9 to about 12 hours, about 10 to about 12 hours, about 4 to about 11 hours, about 5 to about 11 hours, about 6 to about 11 hours, about 7 to about 11 hours, about 8 to about 11 hours, about 4 to about 10 hours, about 5 to about 10 hours, about 6 to about 10 hours, about 7 to about 10 hours, about 8 to about 10 hours or about 4 to about 6 hours. In an embodiment, the IV infusion may be carried for about 4 hours. In an embodiment, the IV infusion may be carried for about 6 hours. In an embodiment, the IV infusion may be carried for about 8 hours. In an embodiment, the IV infusion may be carried for about 9 hours. In an embodiment, the IV infusion may be carried for about 10 hours. In an embodiment, the IV infusion may be carried for about 11 hours.

According to an embodiment, the compound of Formula (I) or a salt thereof could be administered as a fixed dose or based on bodyweight of a subject.

The compound of Formula (I) or a salt thereof may be administered at an effective dose of from about 500 mg to about 2,000 mg (calculated as 4-butoxy-N-((4-(dimethylamino)tetrahydro-2H-pyran-4-yl)methyl)-3,5-dimethoxybenzamide hydrochloride (MW 430.97), unless indicated otherwise). In an embodiment, the effective dose may be in a range from about 600 mg to about 1,900 mg, about 600 mg to about 1,800 mg, about 600 mg to about 1,700 mg, about 600 mg to about 1,600 mg, about 600 mg to about 1,500 mg, about 600 mg to about 1,400 mg, about 600 mg to about 1,300 mg, about 600 mg to about 1,200 mg, about 600 mg to about 1,100 mg, about 600 mg to about 1000 mg, about 600 mg to about 900 mg, about 700 mg to about 1,900 mg, about 700 mg to about 1,800 mg, about 700 mg to about 1,700 mg, about 700 mg to about 1,600 mg, about 700 mg to about 1,500 mg, about 700 mg to about 1,400 mg, about 700 mg to about 1,300 mg, about 700 mg to about 1,200 mg, about 700 mg to about 1,100 mg, about 700 mg to about 1000 mg, about 700 mg to about 900 mg, about 800 mg to about 1,900 mg, about 800 mg to about 1,800 mg, about 800 mg to about 1,700 mg, about 800 mg to about 1,600 mg, about 800 mg to about 1,500 mg, about 800 mg to about 1,400 mg, about 800 mg to about 1,300 mg, about 800 mg to about 1,200 mg, about 800 mg to about 1,100 mg, about 800 mg to about 1000 mg, about 800 mg to about 900 mg, about 900 mg to about 1,900 mg, about 900 mg to about 1,800 mg, about 900 mg to about 1,700 mg, about 900 mg to about 1,600 mg, about 900 mg to about 1,500 mg, about 900 mg to about 1,400 mg, about 900 mg to about 1,300 mg, about 900 mg to about 1,200 mg, about 900 mg to about 1,100 mg, about 900 mg to about 1000 mg, about 1,000 mg to about 1,900 mg, about 1,000 mg to about 1,800 mg, about 1,000 mg to about 1,700 mg, about 1,000 mg to about 1,600 mg, about 1,000 mg to about 1,500 mg, about 1,000 mg to about 1,400 mg, about 1,000 mg to about 1,300 mg, about 1,000 mg to about 1,200 mg, or about 1,000 mg to about 1,100 mg.

In another embodiment, the effective dose may be about 5 mg/kg bodyweight to about 35 mg/kg bodyweight. In an embodiment, the effective dose may range from about 8 mg/kg to about 25 mg/kg, about 9 mg/kg to about 25 mg/kg, about 10 mg/kg to about 25 mg/kg, about 11 mg/kg to about 25 mg/kg, about 12 mg/kg to about 25 mg/kg, about 13 mg/kg to about 25 mg/kg, about 14 mg/kg to about 25 mg/kg, about 15 mg/kg to about 25 mg/kg, about 16 mg/kg to about 25 mg/kg, about 17 mg/kg to about 25 mg/kg, about 18 mg/kg to about 25 mg/kg, about 19 mg/kg to about 25 mg/kg, about 20 mg/kg to about 25 mg/kg, about 8 mg/kg to about 23 mg/kg, about 9 mg/kg to about 23 mg/kg, about 10 mg/kg to about 23 mg/kg, about 11 mg/kg to about 23 mg/kg, about 12 mg/kg to about 23 mg/kg, about 13 mg/kg to about 23 mg/kg, about 14 mg/kg to about 23 mg/kg, about 15 mg/kg to about 23 mg/kg, about 16 mg/kg to about 23 mg/kg, about 17 mg/kg to about 23 mg/kg, about 18 mg/kg to about 23 mg/kg, about 19 mg/kg to about 23 mg/kg, about 20 mg/kg to about 23 mg/kg, about 8 mg/kg to about 20 mg/kg, about 9 mg/kg to about 20 mg/kg, about 10 mg/kg to about 20 mg/kg, about 11 mg/kg to about 20 mg/kg, about 12 mg/kg to about 20 mg/kg, about 13 mg/kg to about 20 mg/kg, about 14 mg/kg to about 20 mg/kg, about 15 mg/kg to about 20 mg/kg, about 16 mg/kg to about 20 mg/kg, about 17 mg/kg to about 20 mg/kg, about 18 mg/kg to about 20 mg/kg, about 8 mg/kg to about 19 mg/kg, about 9 mg/kg to about 19 mg/kg, about 10 mg/kg to about 19 mg/kg, about 11 mg/kg to about 19 mg/kg, about 12 mg/kg to about 19 mg/kg, about 13 mg/kg to about 19 mg/kg, about 14 mg/kg to about 19 mg/kg, about 15 mg/kg to about 19 mg/kg, about 16 mg/kg to about 19 mg/kg, about 17 mg/kg to about 19 mg/kg, about 8 mg/kg to about 18 mg/kg, about 9 mg/kg to about 18 mg/kg, about 10 mg/kg to about 18 mg/kg, about 11 mg/kg to about 18 mg/kg, about 12 mg/kg to about 18 mg/kg, about 13 mg/kg to about 18 mg/kg, about 14 mg/kg to about 18 mg/kg, about 15 mg/kg to about 18 mg/kg, about 16 mg/kg to about 18 mg/kg, about 8 mg/kg to about 17 mg/kg, about 9 mg/kg to about 17 mg/kg, about 10 mg/kg to about 17 mg/kg, about 11 mg/kg to about 17 mg/kg, about 12 mg/kg to about 17 mg/kg, about 13 mg/kg to about 17 mg/kg, about 14 mg/kg to about 17 mg/kg, about 15 mg/kg to about 17 mg/kg, or about 16 mg/kg to about 17 mg/kg.

In an embodiment, the effective dose may be in a range from about 11 mg/kg to about 18.5 mg/kg, 11.5 mg/kg to about 18.5 mg/kg, about 12 mg/kg to about 18.5 mg/kg, about 12.5 mg/kg to about 18.5 mg/kg, about 13 mg/kg to about 18.5 mg/kg, from about 13.5 mg/kg to about 18.5 mg/kg, from about 14 mg/kg to about 18.5 mg/kg, from about 14.5 mg/kg to about 18.5 mg/kg, from about 15 mg/kg to about 18.5 mg/kg, from about 15.5 mg/kg to about 18.5 mg/kg, from about 16 mg/kg to about 18.5 mg/kg, about 11 mg/kg to about 17.5 mg/kg, 11.5 mg/kg to about 17.5 mg/kg, about 12 mg/kg to about 17.5 mg/kg, about 12.5 mg/kg to about 17.5 mg/kg, about 13 mg/kg to about 17.5 mg/kg, from about 13.5 mg/kg to about 17.5 mg/kg, from about 14 mg/kg to about 17.5 mg/kg, from about 14.5 mg/kg to about 17.5 mg/kg, from about 15 mg/kg to about 17.5 mg/kg, from about 15.5 mg/kg to about 17.5 mg/kg, or from about 16 mg/kg to about 17.5 mg/kg.

The dosing can be stepwise, tapered, or increased over the administration period. In an exemplary embodiment, when the total administration duration is about 10 hrs and the effective dose is about 1,000 mg, the initial dose may be about 160 mg of the compound for about 0.3-about 1 hrs, followed by a maintenance dose of 840 mg for about 7-about 9.7 hrs. In another exemplary embodiment, the initial loading dose may be about 1-2.5 mg/kg for about 0.3-about 1 hrs, followed by a maintenance dose of about 0.8-1.8 mg/kg for about 7-about 9.7 hrs.

In an exemplary embodiment, yet another embodiment, patients underwent operation and received the compound of Formula (I) reported a significant reduction in intensity of pain as well as the total amount of postoperative analgesia consumed during the first 24 hours post emergence.

In a further embodiment, the moderate to severe pain is reported by the subject to have a pain intensity (PI) score of 4 or greater, 5 or greater, 6 or greater, or 7 or greater. The reported pain is pain experienced by the subject prior to administration of the compound of Formula (I). In particular embodiments, the PI may be 7, 8, 9, or 10.

In an embodiment, the method according to the aspect may be applied to a subject who reports a previous experience of moderate to severe postoperative pain.

Treatment To Reduce the Amount of Postoperative Analgesia Administered

In an embodiment, the preoperative pain treatment includes reducing an amount of an analgesic administered during and/or after an operation to treat postoperative pain of a subject, comprising administering to subject a pharmaceutically effective amount of a compound of Formula (I) up to about 10 hours, wherein the administration is carried out continuous infusion, to the subject. In an embodiment, the analgesic is an opioid, an NSAID, or its combination.

Various opioid analgesics are known and described in more detail hereinafter.

In an exemplary embodiment, the method of reducing the amount of postoperative analgesia administered to a patient who underwent operation includes administering a compound of Formula (I) or a salt thereof prior to the operation (“preoperative dosing” or “preoperative administration”); commencing the administration prior to the operation (for example, about 1 hr—about 30 minutes prior to anesthesia induction) and continues during the operation and ends during the operation or after the operation) (“pre-, intra- and post-operation dosing” or “pre- and intra-operation dosing”); during the operation (“intra-operation dosing”); commencing the administration during the operation or before emergence from anesthesia and continuing it after the operation (“intra- and post-operation dosing”); or after emergence from the operation.

The administration may be carried out continuously by IV infusion for about 5 hours to about 15 hours. In another embodiment, the IV infusion could be carried out for about 6 to about 15 hours, about 7 to about 15 hours, about 8 to about 15 hours, about 9 to about 15 hours, about 10 to about 15 hours, about 5 to about 12 hours, about 6 to about 12 hours, about 7 to about 12 hours, about 8 to about 12 hours, about 9 to about 12 hours, about 10 to about 12 hours, about 5 to about 11 hours, about 6 to about 11 hours, about 7 to about 11 hours, about 8 to about 11 hours, about 5 to about 10 hours, about 6 to about 10 hours, about 7 to about 10 hours, or about 8 to about 10 hours. In an embodiment, the IV infusion may be carried for about 8 hours. In an embodiment, the IV infusion may be carried for about 9 hours. In an embodiment, the IV infusion may be carried for about 10 hours. In an embodiment, the IV infusion may be carried for about 11 hours.

According to an embodiment, the compound of Formula (I) or a salt thereof could be administered as a fixed dose or based on bodyweight of a subject.

The compound of Formula (I) or a salt thereof may be administered at an effective dose of from about 500 mg to about 2,000 mg (calculated as 4-butoxy-N14(4-(dimethylamino)tetrahydro-2H-pyran-4-yl)methyl)-3,5-dimethoxybenzamide hydrochloride (MW 430.97), unless indicated otherwise). In an embodiment, the effective dose may be in a range from about 600 mg to about 1,900 mg, about 600 mg to about 1,800 mg, about 600 mg to about 1,700 mg, about 600 mg to about 1,600 mg, about 600 mg to about 1,500 mg, about 600 mg to about 1,400 mg, about 600 mg to about 1,300 mg, about 600 mg to about 1,200 mg, about 600 mg to about 1,100 mg, about 600 mg to about 1000 mg, about 600 mg to about 900 mg, about 700 mg to about 1,900 mg, about 700 mg to about 1,800 mg, about 700 mg to about 1,700 mg, about 700 mg to about 1,600 mg, about 700 mg to about 1,500 mg, about 700 mg to about 1,400 mg, about 700 mg to about 1,300 mg, about 700 mg to about 1,200 mg, about 700 mg to about 1,100 mg, about 700 mg to about 1000 mg, about 700 mg to about 900 mg, about 800 mg to about 1,900 mg, about 800 mg to about 1,800 mg, about 800 mg to about 1,700 mg, about 800 mg to about 1,600 mg, about 800 mg to about 1,500 mg, about 800 mg to about 1,400 mg, about 800 mg to about 1,300 mg, about 800 mg to about 1,200 mg, about 800 mg to about 1,100 mg, about 800 mg to about 1000 mg, about 800 mg to about 900 mg, about 900 mg to about 1,900 mg, about 900 mg to about 1,800 mg, about 900 mg to about 1,700 mg, about 900 mg to about 1,600 mg, about 900 mg to about 1,500 mg, about 900 mg to about 1,400 mg, about 900 mg to about 1,300 mg, about 900 mg to about 1,200 mg, about 900 mg to about 1,100 mg, about 900 mg to about 1000 mg, about 1,000 mg to about 1,900 mg, about 1,000 mg to about 1,800 mg, about 1,000 mg to about 1,700 mg, about 1,000 mg to about 1,600 mg, about 1,000 mg to about 1,500 mg, about 1,000 mg to about 1,400 mg, about 1,000 mg to about 1,300 mg, about 1,000 mg to about 1,200 mg, or about 1,000 mg to about 1,100 mg.

In another embodiment, the effective dose may be about 5 mg/kg bodyweight to about 35 mg/kg bodyweight. In an embodiment, the effective dose may range from about 8 mg/kg to about 25 mg/kg, about 9 mg/kg to about 25 mg/kg, about 10 mg/kg to about 25 mg/kg, about 11 mg/kg to about 25 mg/kg, about 12 mg/kg to about 25 mg/kg, about 13 mg/kg to about 25 mg/kg, about 14 mg/kg to about 25 mg/kg, about 15 mg/kg to about 25 mg/kg, about 16 mg/kg to about 25 mg/kg, about 17 mg/kg to about 25 mg/kg, about 18 mg/kg to about 25 mg/kg, about 19 mg/kg to about 25 mg/kg, about 20 mg/kg to about 25 mg/kg, about 8 mg/kg to about 23 mg/kg, about 9 mg/kg to about 23 mg/kg, about 10 mg/kg to about 23 mg/kg, about 11 mg/kg to about 23 mg/kg, about 12 mg/kg to about 23 mg/kg, about 13 mg/kg to about 23 mg/kg, about 14 mg/kg to about 23 mg/kg, about 15 mg/kg to about 23 mg/kg, about 16 mg/kg to about 23 mg/kg, about 17 mg/kg to about 23 mg/kg, about 18 mg/kg to about 23 mg/kg, about 19 mg/kg to about 23 mg/kg, about 20 mg/kg to about 23 mg/kg, about 8 mg/kg to about 20 mg/kg, about 9 mg/kg to about 20 mg/kg, about 10 mg/kg to about 20 mg/kg, about 11 mg/kg to about 20 mg/kg, about 12 mg/kg to about 20 mg/kg, about 13 mg/kg to about 20 mg/kg, about 14 mg/kg to about 20 mg/kg, about 15 mg/kg to about 20 mg/kg, about 16 mg/kg to about 20 mg/kg, about 17 mg/kg to about 20 mg/kg, about 18 mg/kg to about 20 mg/kg, about 8 mg/kg to about 19 mg/kg, about 9 mg/kg to about 19 mg/kg, about 10 mg/kg to about 19 mg/kg, about 11 mg/kg to about 19 mg/kg, about 12 mg/kg to about 19 mg/kg, about 13 mg/kg to about 19 mg/kg, about 14 mg/kg to about 19 mg/kg, about 15 mg/kg to about 19 mg/kg, about 16 mg/kg to about 19 mg/kg, about 17 mg/kg to about 19 mg/kg, about 8 mg/kg to about 18 mg/kg, about 9 mg/kg to about 18 mg/kg, about 10 mg/kg to about 18 mg/kg, about 11 mg/kg to about 18 mg/kg, about 12 mg/kg to about 18 mg/kg, about 13 mg/kg to about 18 mg/kg, about 14 mg/kg to about 18 mg/kg, about 15 mg/kg to about 18 mg/kg, about 16 mg/kg to about 18 mg/kg, about 8 mg/kg to about 17 mg/kg, about 9 mg/kg to about 17 mg/kg, about 10 mg/kg to about 17 mg/kg, about 11 mg/kg to about 17 mg/kg, about 12 mg/kg to about 17 mg/kg, about 13 mg/kg to about 17 mg/kg, about 14 mg/kg to about 17 mg/kg, about 15 mg/kg to about 17 mg/kg, or about 16 mg/kg to about 17 mg/kg.

In an embodiment, the effective dose may be in a range from about 11 mg/kg to about 18.5 mg/kg, 11.5 mg/kg to about 18.5 mg/kg, about 12 mg/kg to about 18.5 mg/kg, about 12.5 mg/kg to about 18.5 mg/kg, about 13 mg/kg to about 18.5 mg/kg, from about 13.5 mg/kg to about 18.5 mg/kg, from about 14 mg/kg to about 18.5 mg/kg, from about 14.5 mg/kg to about 18.5 mg/kg, from about 15 mg/kg to about 18.5 mg/kg, from about 15.5 mg/kg to about 18.5 mg/kg, from about 16 mg/kg to about 18.5 mg/kg, about 11 mg/kg to about 17.5 mg/kg, 11.5 mg/kg to about 17.5 mg/kg, about 12 mg/kg to about 17.5 mg/kg, about 12.5 mg/kg to about 17.5 mg/kg, about 13 mg/kg to about 17.5 mg/kg, from about 13.5 mg/kg to about 17.5 mg, from about 14 mg/kg to about 17.5 mg/kg, from about 14.5 mg/kg to about 17.5 mg/kg, from about 15 mg/kg to about 17.5 mg/kg, from about 15.5 mg/kg to about 17.5 mg/kg, or from about 16 mg/kg to about 17.5 mg/kg.

The dosing can be stepwise, tapered, or increased over the administration period. In an exemplary embodiment, when the total administration duration is about 10 hrs and the effective dose is about 1,000 mg, the initial dose may be about 160 mg of the compound for about 0.3-about 1 hrs, followed by a maintenance dose of 840 mg for about 7-about 9.7 hrs. In another exemplary embodiment, the initial loading dose may be about 1-2.5 mg/kg for about 0.3-about 1 hrs, followed by a maintenance dose of about 0.8-1.8 mg/kg for about 7-about 9.7 hrs.

In an embodiment, the postoperative pain is moderate to severe pain. In an aspect, the moderate to severe pain is reported by the subject to have a pain intensity (PI) score of 4 or greater, 5 or greater, 6 or greater, or 7 or greater. The reported pain may be pain experienced by the subject prior to administration of the compound of Formula (I). For example, the pain intensity may be reported at or during first hour after emergence from anesthesia. In particular embodiments, the PI may be 7, 8, 9, or 10.

In yet another embodiment, the total amount of the opioid administered as postoperative pain treatment during the first 24 hours post emergence is reduced by at least 30% when compared to the total amount consumed by a placebo patient.

### Treatment to Reduce Need of Postoperative Analgesia

One aspect of the present invention is directed to a method for reducing need of a postoperative analgesia by a subject suffering from postoperative pain, comprising administering to the subject an effective amount of a compound of Formula (I) or pharmaceutically acceptable salt thereof, wherein the postoperative analgesia comprises a subject self-administered analgesia, a rescue analgesia, or both, and wherein the need of postoperative analgesia comprises a frequency of patient self-administration; an amount of self-administered analgesia; a frequency of rescue request; and/or a total amount of administered postoperative analgesia. In an embodiment, the need of postoperative analgesia is during first about 72 hours after emergence from anesthesia. In an embodiment, the need of postoperative analgesia is during first about 60 hours, first about 48 hours, first about 36 hours, about 24 hours after emergence from anesthesia.

In an embodiment, the subject is human patient. In an aspect, the human patient has a capacity of self-administering an analgesia. Self-administration may be carried out via a patient-controlled analgesia (PCA). In an embodiment, the self-administered analgesia is an opioid. In an embodiment, the rescue analgesia may be an opioid. Exemplary non-limiting opioid may include morphine, codeine, fentanyl transdermal, hydrocodone, hydromorphone, oxycodone, oxymorphone, tapentadol, tramadol, or the like.

In an embodiment, the patient is those who reported pain intensity score of 4 or greater, 5 or greater, 6 or greater, 7 or greater, at the emergence from anesthesia. In another embodiment, the patient is those who reported pain intensity score of 7 or greater at the emergence from anesthesia. In another embodiment, the patient is those who reported pain intensity score of 4 or greater, 5 or greater, 6 or greater, 7 or greater at the emergence from anesthesia and requested a rescue analgesia during first 2 hours after emergence from anesthesia. In some embodiment, the rescue request may be made during first 1 hour after emergence from anesthesia.

In an exemplary embodiment, the method of reducing the amount of postoperative analgesia administered to a patient who underwent operation includes administering a compound of Formula (I) or a salt thereof prior to the operation (“preoperative dosing” or “preoperative administration”); commencing the administration prior to the operation (for example, about 1 hr—about 30 minutes prior to anesthesia induction) and continues during the operation and ends during the operation or after the operation) (“pre-, intra- and post-operation dosing” or “pre- and intra-operation dosing”); during the operation (“intra-operation dosing”); commencing the administration during the operation or before emergence from anesthesia and continuing it after the operation (“intra- and post-operation dosing”); or after emergence from the operation.

In an embodiment, the compound of Formula (I) is administered before, during or after an operation to treat postoperative pain in the subject. In a particular embodiment, the compound of Formula (I) is administered after an operation. In yet another embodiment, the compound of Formula (I) is administered during an operation.

The administration may be carried out continuously by IV infusion for about 5 hours to about 15 hours. In another embodiment, the IV infusion could be carried out for about 6 to about 15 hours, about 7 to about 15 hours, about 8 to about 15 hours, about 9 to about 15 hours, about 10 to about 15 hours, about 5 to about 12 hours, about 6 to about 12 hours, about 7 to about 12 hours, about 8 to about 12 hours, about 9 to about 12 hours, about 10 to about 12 hours, about 5 to about 11 hours, about 6 to about 11 hours, about 7 to about 11 hours, about 8 to about 11 hours, about 5 to about 10 hours, about 6 to about 10 hours, about 7 to about 10 hours, or about 8 to about 10 hours. In an embodiment, the IV infusion may be carried for about 8 hours. In an embodiment, the IV infusion may be carried for about 9 hours. In an embodiment, the IV infusion may be carried for about 10 hours. In an embodiment, the IV infusion may be carried for about 11 hours.

Accordingly, in exemplary embodiments, administering of the compound of Formula (I) starts about 1 hour to 30 minutes before completion of the operation. In yet another embodiment, administering of the compound of Formula (I) starts about 1 hour to 30 minutes before completion of the operation, and the compound of Formula (I) is further administered for about 0.9 to 9.5 hours after the operation. In yet another embodiment, the compound of Formula (I) is administered before the start of an operation to treat postoperative pain of a subject. In a particular embodiment, the compound of Formula (I) is administered before induction of anesthesia. In yet another embodiment, administering of the compound of Formula (I) starts about 1 hour to 20 minutes before the operation. In yet another embodiment, administering of the compound of Formula (I) starts about 1 hour to 20 minutes before the operation, and the compound of Formula (I) is further administered during the operation. In another embodiment, a first dose of the compound of Formula (I) is administered over the course of 30 minutes prior to induction of anesthesia. In yet a further embodiment, a second dose of the compound is administered over about 9.5 hours following completion of administration of the first dose.

According to an embodiment, the compound of Formula (I) or a salt thereof could be administered as a fixed dose or based on bodyweight of a subject.

The compound of Formula (I) or a salt thereof may be administered at an effective dose of from about 500 mg to about 2,000 mg (calculated as 4-butoxy-N-((4-(dimethylamino)tetrahydro-2H-pyran-4-yl)methyl)-3,5-dimethoxybenzamide hydrochloride (MW 430.97), unless indicated otherwise). In an embodiment, the effective dose may be in a range from about 600 mg to about 1,900 mg, about 600 mg to about 1,800 mg, about 600 mg to about 1,700 mg, about 600 mg to about 1,600 mg, about 600 mg to about 1,500 mg, about 600 mg to about 1,400 mg, about 600 mg to about 1,300 mg, about 600 mg to about 1,200 mg, about 600 mg to about 1,100 mg, about 600 mg to about 1000 mg, about 600 mg to about 900 mg, about 700 mg to about 1,900 mg, about 700 mg to about 1,800 mg, about 700 mg to about 1,700 mg, about 700 mg to about 1,600 mg, about 700 mg to about 1,500 mg, about 700 mg to about 1,400 mg, about 700 mg to about 1,300 mg, about 700 mg to about 1,200 mg, about 700 mg to about 1,100 mg, about 700 mg to about 1000 mg, about 700 mg to about 900 mg, about 800 mg to about 1,900 mg, about 800 mg to about 1,800 mg, about 800 mg to about 1,700 mg, about 800 mg to about 1,600 mg, about 800 mg to about 1,500 mg, about 800 mg to about 1,400 mg, about 800 mg to about 1,300 mg, about 800 mg to about 1,200 mg, about 800 mg to about 1,100 mg, about 800 mg to about 1000 mg, about 800 mg to about 900 mg, about 900 mg to about 1,900 mg, about 900 mg to about 1,800 mg, about 900 mg to about 1,700 mg, about 900 mg to about 1,600 mg, about 900 mg to about 1,500 mg, about 900 mg to about 1,400 mg, about 900 mg to about 1,300 mg, about 900 mg to about 1,200 mg, about 900 mg to about 1,100 mg, about 900 mg to about 1000 mg, about 1,000 mg to about 1,900 mg, about 1,000 mg to about 1,800 mg, about 1,000 mg to about 1,700 mg, about 1,000 mg to about 1,600 mg, about 1,000 mg to about 1,500 mg, about 1,000 mg to about 1,400 mg, about 1,000 mg to about 1,300 mg, about 1,000 mg to about 1,200 mg, or about 1,000 mg to about 1,100 mg.

In another embodiment, the effective dose may be about 5 mg/kg bodyweight to about 35 mg/kg bodyweight. In an embodiment, the effective dose may range from about 8 mg/kg to about 25 mg/kg, about 9 mg/kg to about 25 mg/kg, about 10 mg/kg to about 25 mg/kg, about 11 mg/kg to about 25 mg/kg, about 12 mg/kg to about 25 mg/kg, about 13 mg/kg to about 25 mg/kg, about 14 mg/kg to about 25 mg/kg, about 15 mg/kg to about 25 mg/kg, about 16 mg/kg to about 25 mg/kg, about 17 mg/kg to about 25 mg/kg, about 18 mg/kg to about 25 mg/kg, about 19 mg/kg to about 25 mg/kg, about 20 mg/kg to about 25 mg/kg, about 8 mg/kg to about 23 mg/kg, about 9 mg/kg to about 23 mg/kg, about 10 mg/kg to about 23 mg/kg, about 11 mg/kg to about 23 mg/kg, about 12 mg/kg to about 23 mg/kg, about 13 mg/kg to about 23 mg/kg, about 14 mg/kg to about 23 mg/kg, about 15 mg/kg to about 23 mg/kg, about 16 mg/kg to about 23 mg/kg, about 17 mg/kg to about 23 mg/kg, about 18 mg/kg to about 23 mg/kg, about 19 mg/kg to about 23 mg/kg, about 20 mg/kg to about 23 mg/kg, about 8 mg/kg to about 20 mg/kg, about 9 mg/kg to about 20 mg/kg, about 10 mg/kg to about 20 mg/kg, about 11 mg/kg to about 20 mg/kg, about 12 mg/kg to about 20 mg/kg, about 13 mg/kg to about 20 mg/kg, about 14 mg/kg to about 20 mg/kg, about 15 mg/kg to about 20 mg/kg, about 16 mg/kg to about 20 mg/kg, about 17 mg/kg to about 20 mg/kg, about 18 mg/kg to about 20 mg/kg, about 8 mg/kg to about 19 mg/kg, about 9 mg/kg to about 19 mg/kg, about 10 mg/kg to about 19 mg/kg, about 11 mg/kg to about 19 mg/kg, about 12 mg/kg to about 19 mg/kg, about 13 mg/kg to about 19 mg/kg, about 14 mg/kg to about 19 mg/kg, about 15 mg/kg to about 19 mg/kg, about 16 mg/kg to about 19 mg/kg, about 17 mg/kg to about 19 mg/kg, about 8 mg/kg to about 18 mg/kg, about 9 mg/kg to about 18 mg/kg, about 10 mg/kg to about 18 mg/kg, about 11 mg/kg to about 18 mg/kg, about 12 mg/kg to about 18 mg/kg, about 13 mg/kg to about 18 mg/kg, about 14 mg/kg to about 18 mg/kg, about 15 mg/kg to about 18 mg/kg, about 16 mg/kg to about 18 mg/kg, about 8 mg/kg to about 17 mg/kg, about 9 mg/kg to about 17 mg/kg, about 10 mg/kg to about 17 mg/kg, about 11 mg/kg to about 17 mg/kg, about 12 mg/kg to about 17 mg/kg, about 13 mg/kg to about 17 mg/kg, about 14 mg/kg to about 17 mg/kg, about 15 mg/kg to about 17 mg/kg, or about 16 mg/kg to about 17 mg/kg.

In an embodiment, the effective dose may be in a range from about 11 mg/kg to about 18.5 mg/kg, 11.5 mg/kg to about 18.5 mg/kg, about 12 mg/kg to about 18.5 mg/kg, about 12.5 mg/kg to about 18.5 mg/kg, about 13 mg/kg to about 18.5 mg/kg, from about 13.5 mg/kg to about 18.5 mg/kg, from about 14 mg/kg to about 18.5 mg/kg, from about 14.5 mg/kg to about 18.5 mg/kg, from about 15 mg/kg to about 18.5 mg/kg, from about 15.5 mg/kg to about 18.5 mg/kg, from about 16 mg/kg to about 18.5 mg/kg, about 11 mg/kg to about 17.5 mg/kg, 11.5 mg/kg to about 17.5 mg/kg, about 12 mg/kg to about 17.5 mg/kg, about 12.5 mg/kg to about 17.5 mg/kg, about 13 mg/kg to about 17.5 mg/kg, from about 13.5 mg/kg to about 17.5 mg/kg, from about 14 mg/kg to about 17.5 mg/kg, from about 14.5 mg/kg to about 17.5 mg/kg, from about 15 mg/kg to about 17.5 mg/kg, from about 15.5 mg/kg to about 17.5 mg/kg, or from about 16 mg/kg to about 17.5 mg/kg.

The dosing can be stepwise, tapered, or increased over the administration period. In an exemplary embodiment, when the total administration duration is about 10 hrs and the effective dose is about 1,000 mg, the initial dose may be about 160 mg of the compound for about 0.3-about 1 hrs, followed by a maintenance dose of 840 mg for about 7-about 9.7 hrs. In another exemplary embodiment, the initial loading dose may be about 1-2.5 mg/kg for about 0.3-about 1 hrs, followed by a maintenance dose of about 0.8-1.8 mg/kg for about 7-about 9.7 hrs. In yet a further embodiment, the first dose is about 100 to 200 mg and the second dose is about 700 mg to 1,000 mg.

In a further embodiment, as result of reduction of the need of postoperative analgesia, the amount of opioid administered during and/or after treatment with a compound of Formula (I) is reduced by 30%, 40%, 50%, 60%, 70%, 80%, 90%, or 100% when compared to an amount consumed by a placebo patient. In another embodiment, the amount of opioid administered after treatment with a compound of Formula (I) is reduced 30%-100%, 40%-100%, 50%-100%, 60%-100%, 70%-100%, 80%-100%, 90%-100%, or 95%-100% when compared to an amount consumed by a placebo patient. In yet another embodiment, the amount of the opioid administered as needed (i.e., PCA (patient-controlled analgesia, patient's self-administered analgesia), rescue analgesia, or a sum of PCA plus rescue) after the operation is reduced by 30%-90%, 30%-80%, 30%-70%, 30%-60%, 30%-50%, 30%-40%, 35%-85%, 35%-75%, 35%-65%, 35%-55%, or 35%-45% when compared to an amount consumed by a placebo patient.

In yet another embodiment, the frequency of self-administration of pain medication (e.g., opioids) such as patient-controlled analgesia (PCA) is reduced when compared to a number used by a placebo patient. In some embodiments, the frequency of self-administration is reduced by at least 10%, at least 20%, at least 30%, at least 40%, at least 50%, at least 60%, at least 70%, at least 80%, or at least 90%. In some embodiments, the subject may terminate self-administration after the treatment according to an aspect of the present invention.

In a further embodiment, the subject is a patient who experiences the affective-motivational component of pain. The method of the present disclosure may be more effective with a patient experiencing the affective-motivational component of pain than with the other patients. In yet a further embodiment, the subject request rescue medication after emerging from anesthesia, and wherein the rescue medication is in addition to self-administered pain medication (e.g., opioids). In yet a further embodiment, the request for rescue medication is made within the first 2 hours after emergence from anesthesia.

In a further embodiment, a need of an opioid by a subject suffering from postoperative pain is reduced. In yet another embodiment, the opioid is patient-controlled analgesia (PCA) or rescue analgesia provided as needed. In yet another embodiment, the amount of the opioid administered is reduced by at least about 30% when compared to the amount consumed by a placebo patient. In yet another embodiment, the number of demand for the opioid via PCA is reduced by about 50% when compared to a placebo patient.

In another embodiment, the subject is in negative affective states. In some embodiments, the patient treated by the compound of this disclosure may a patient having a high level of anxiety, pain catastrophizing or pain intensity. The method of the present disclosure may be more effective with a patient having a high level of anxiety, pain catastrophizing, or pain intensity than with the other patients. Such patients are prone to use more amounts of PCA when compared to other patients. Administration of the compound is effective to reduce an amount of opioid used for pain treatment, for example, frequencies and/or amounts of PCA. In yet another embodiment, the subject treated by the compound of this disclosure is vulnerable to excessive use of opioid analgesics for pain control. In yet another embodiment, the subject exhibits a genetic variation/polymorphism associated with the metabolism and effects of opioids. In yet another embodiment, the genetic variation includes mu-opioid receptors OPRM1 gene variant, serotonin receptors 5HT2A gene variant, or GCH1 gene variant for the tetrahydrobiopterin synthesis pathway. Individual differences in self-reported pain intensity illustrate the multifaceted nature of pain, which has evolved as a behavioral repertoire that promotes survival and adaptive action in each organism. The subjective experience of pain in particular activates defensive motivational systems (e.g. avoidance or escape) against somatic threats from which negative affect concurrently emanates, such as fear and anxiety, pain catastrophizing, and depression. This affective-motivational domain of pain may involve a phenotypic vulnerability and/or a genetic predisposition to physical and emotional stressors, which have often been attributed to differential activations of endogenous opioid and dopamine systems. For postoperative pain, pain sensitivity and/or postoperative opioid use have also been associated with several candidate polymorphisms in mu-opioid receptors (OPRM137,45), serotonin antagonism (5HT2AR2), and tetrahydrobiopterin synthesis (BH4/GCH190). For example, patients with OPRM1 118G-carriers (A118G, rs1799971) were more susceptible to higher levels of opioid consumption after total knee arthroplasty, abdominal hysterectomy or myomectomy. The visual or numeric rating scale of pain intensity currently recommended as the primary evaluation of the efficacy of candidate drugs, however, does not address their differential effects on the sensory and affective domains.

It is contemplated that with respect to the inventive methods for the intravenous administration of a compound of Formula (I) as described herein, other analgesics, for example conventional opioid analgesics, may be used to treat perioperative pain and/or postoperative pain in the patient(s), as well. It is particularly contemplated that one or more opioid analgesics will be administered post-surgically to the patient as rescue medicine in order to treat breakthrough pain that the patient may experience. In various embodiments, the method reduces the amount of conventional opioid analgesics used after surgery by 30%, 40%, 50%, 60%, 70%, 80%, 90%, or 100% when compared to an amount consumed by a placebo patient. In another embodiment, the amount of conventional opioid analgesics administered after surgery is reduced 30%-100%, 40%-100%, 50%-100%, 60%-100%, 70%-100%, 80%-100%, 90%-100%, or 95%-100% when compared to an amount consumed by a placebo patient.

Opioid Analgesic

The term “opioid analgesic” refers to all drugs, natural or synthetic, with morphine-like actions. The synthetic and semi-synthetic opioid analgesics are derivatives of five chemical classes of compound: phenanthrenes; phenylheptylamines; phenylpiperidines; morphinans; and benzomorphans, all of which are within the scope of the term. Opioid analgesics which can be used in one embodiment include all opioid agonists or mixed agonist-antagonists, partial agonists, including but not limited to alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, mixtures of any of the foregoing, salts of any of the foregoing, and the like. In this specification, the term “opioid” and “opioid analgesic” are sometime interchangeably used.

In certain embodiments, opioid analgesics include morphine, oxycodone, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine or pharmaceutically acceptable salts thereof. In certain preferred embodiments, the opioid agonist is morphine. Equianalgesic doses of these opioids are generally known to those persons having ordinary skill in the art.

The following table shows a list of commonly prescribed opioid as postoperative acute pain treatment:

| | Approximate Equianalgesic |
|---|---|
| Opioid | Dose (oral & transdermal)* |
| Morphine | 30 mg |
| (reference) | |
| Codeine | 200 mg |
| Fentanyl | 12.5 mcg/hr |
| transdermal | |
| Hydrocodone | 30 mg |
| Hydromorphone | 7.5 mg |
| Oxycodone | 20 mg |
| Oxymorphone | 10 mg |
| Tapentadol | 75 mg |
| Tramadol | 300 mg |

*Adapted from Von Korff 2008 & FDA labeling

Pain Intensity Score

In certain embodiments, the patient's need for additional analgesic treatment beyond the intravenous administration of a compound of Formula (I) may be ascertained via the use of a surrogate measure of pain. Pain intensity rating scales are used in daily clinical practice to measure pain intensity. The commonly used measurement scales include the Visual Analog Scale (VAS), the Graphic Rating Scale (GRS), the Simple Descriptor Scale (SDS), the Numerical Rating Scale (NRS), and the Faces Rating Scale (FRS). All of these scales have been documented as being valid measures of a pain intensity. The three scales most commonly used in the U.S. are the numerical, word and faces scales. One preferred pain rating scale is the visual analog scale (VAS), a 10 cm vertical or horizontal line with word anchors at the extremes, such as “no pain” on one end and “pain as bad as it could be” on the other. The patient is asked to make a mark along the line to represent pain intensity.

Alternatively, the graphic rating scale (GRS) is a variation of the visual scale which adds words or numbers between the extremes. Wording added might include “no pain”, “mild”, and “severe”. The descriptor scale (SDS) is a list of adjectives describing different levels of pain intensity. For example, pain intensity may be described as “no pain”, “mild”, “moderate”, or “severe”. The numerical pain rating scale (NPRS or NRS) refers to a numerical rating of 0 to 10 or 0 to 5 or to a visual scale with both words and numbers. The patient is asked to rate the pain with 0 being no pain and 10 being the worst possible pain. The faces scale was developed for use with children. This scale exists in several variations but relies on a series of facial expressions to convey pain intensity. Grouping patients' rating of pain intensity as measured with a numerical scale ranging from 0 to 10 into categories of mild, moderate, and severe pain is useful for informing treatment decisions, and interpreting study outcomes. In 1995, Serlin and colleagues (Pain, 1995, 277-84) developed a technique to establish the cut points for mild, moderate, and severe pain by grading pain intensity and functional inference. Since then, a number of studies have been conducted to correlate the numerical scales, for example the NRS, with cutpoints related to levels of pain intensity. Common severity cutpoints are (1 to 4) for mild pain, (5 to 6) for moderate pain, and (7 to 10) for severe pain.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

### EXAMPLES

### Example 1

Sixty subjects aged 18 to 70 underwent laparoscopic colorectal surgery were randomized in a 2:1 ratio to receive either VVZ-149 Injections or a placebo postoperatively. Subjects who reported a pain intensity score of at least 4 on the NRS (0-10) after regaining consciousness in the post-anesthesia care unit (PACU) were enrolled in the study. The VVZ-149 group received an 8-hours IV infusion at a loading dose of 1.8 mg/kg for 0.5 h followed by a maintenance dose of 1.3 mg/kg/h for 7.5 h, and the placebo group received a placebo with the same dosing regimen. All subjects were provided with IV hydromorphone via patient-controlled analgesia (PCA) on demand and rescue dosing provided “as needed” to facilitate adequate pain relief. The primary-outcome measure, pain intensity, was evaluated using NRS (0-10) at the scheduled time points through 24 hours post-emergence. Measures of secondary outcomes included the total request and amount of hydromorphone consumed via PCA and rescue dosing through 24 hours post-emergence.

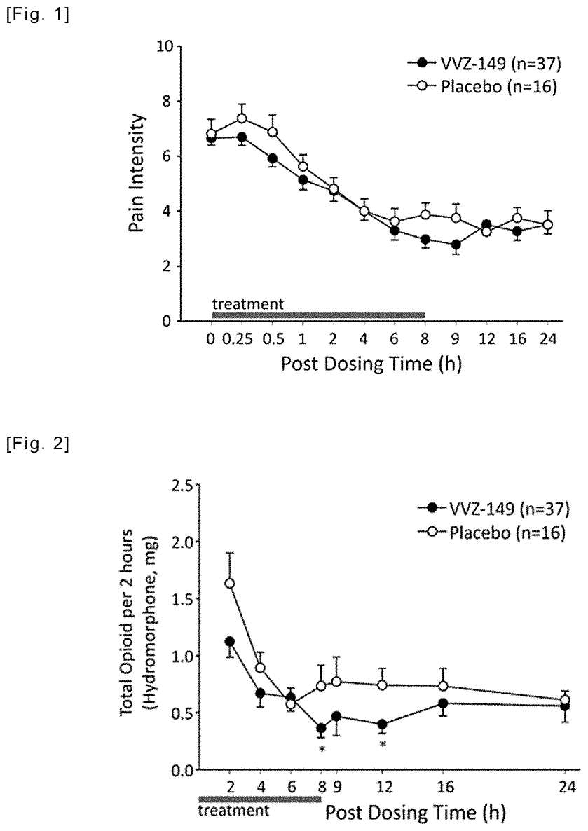

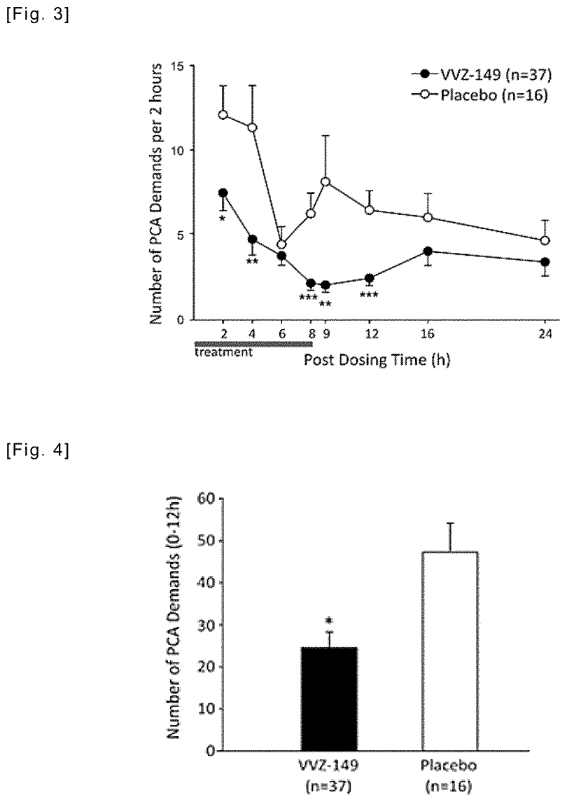

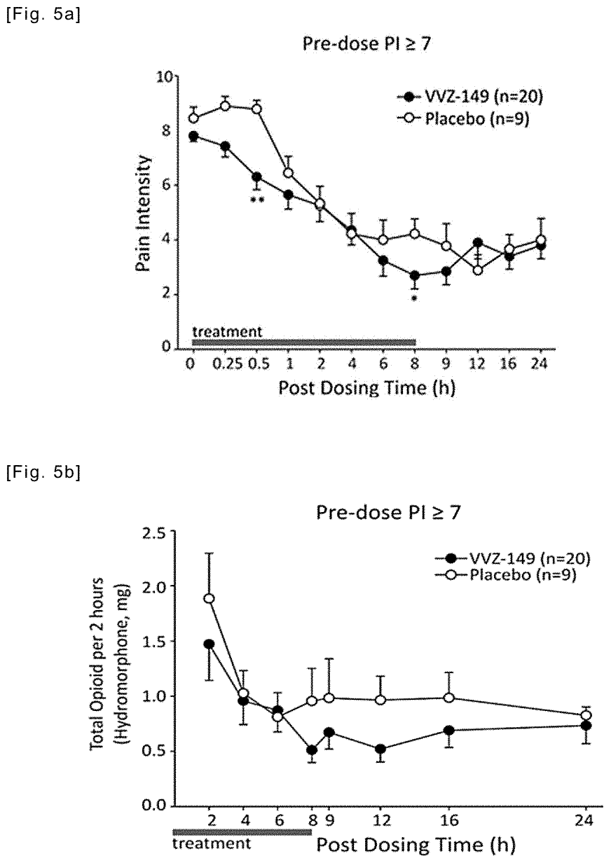

VVZ-149 group showed lower pain intensity with significant less opioid consumption compared to placebo group (FIG. 1, FIG. 2). In particular, the results of number of PCA demand (FIG. 3 and FIG. 4) indicate that VVZ-149 showed significantly reduced the patients' need for postoperative analgesia. In a subgroup with severe postoperative pain (i.e., predose PI 7), significant lower pain intensity was reported and less hydromorphone (via PCA and/or rescue) was requested and consumed in VVZ-149 subgroup compared to placebo subgroup with severe pain (FIG. 5a and FIG. 5b).

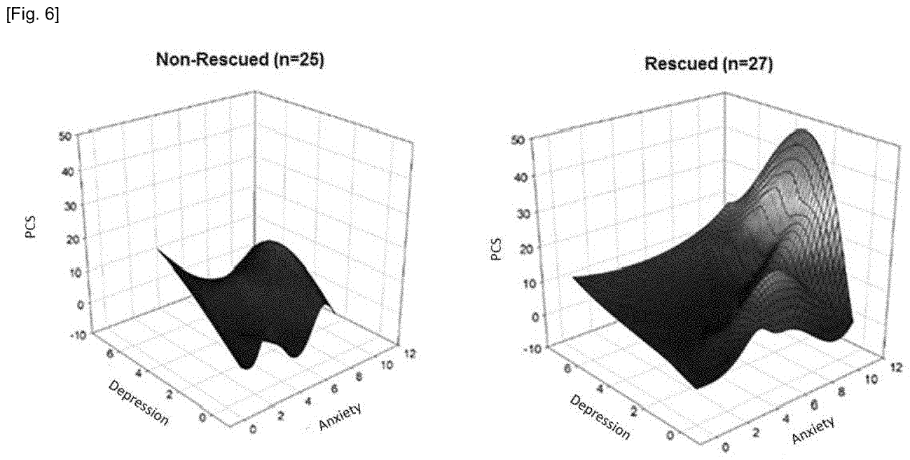

Rescued and non-rescued subgroup was divided for the subgroup of patients who requested additional rescue hydromorphone at least one time in the first 2 hours post-emergence although the PAC was available and who did not. The three-dimensional mesh plot displayed the relationship between anxiety, depression, and pain catastrophizing (PCS) in non-rescued patients and rescued patients (FIG. 6). In rescued patients, anxiety was also highly correlated with both depression (r=0.285, p=0.041) and PCS score (r=0.334, p=0.016). PCS score was also significantly correlated with Predose PI (r=0.389, p=0.045).

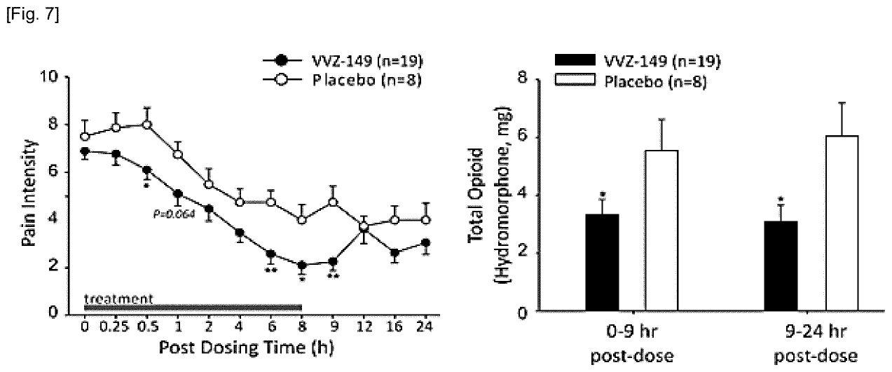

In contrast, non-rescued patients did not show any of these relationships among anxiety, depression, and PCS except the only significant correlation between depression and PCS score (r=0.448, p=0.025). In a subgroup of rescued patients who were more vulnerable to pain, VVZ-149 reduced pain intensity along with a 40% decrease in hydromorphone use compare to placebo same subgroup. Placebo rescued patients did note achieve adequate pain relief despite about twice as much hydromorphone use.(FIG. 7).

### Example 2

Total 307 subjects aged 18 to 70 for an open elective abdominoplasty were randomized in a 1:1 ratio to receive either VVZ-149 Injections or a placebo. The VVZ-149 group received a 10-hours IV infusion starting approximately 30 minutes before the induction of anesthesia at a loading dose of 160 mg for 0.5 h followed by a maintenance dose of 840 mg for 9.5 h, and the placebo group received a placebo with the same dosing regimen. Intraoperative fentanyl was dosed based on objective signs of response to surgical stimulation (increase in HR or SBP>30%) during surgery. All subjects were provided with IV morphine (2 mg of IV, 20-min lockout interval; not to exceed 4 mg in 1.5 hours) on demand as rescue dosing to facilitate adequate pain relief. The primary-outcome measure, pain intensity, was evaluated using NRS (0-10) at the scheduled time points through 48 hours post-emergence. Measures of secondary outcomes included the total request and amount of opioid consumed via rescue dosing through 48 hours post-emergence.

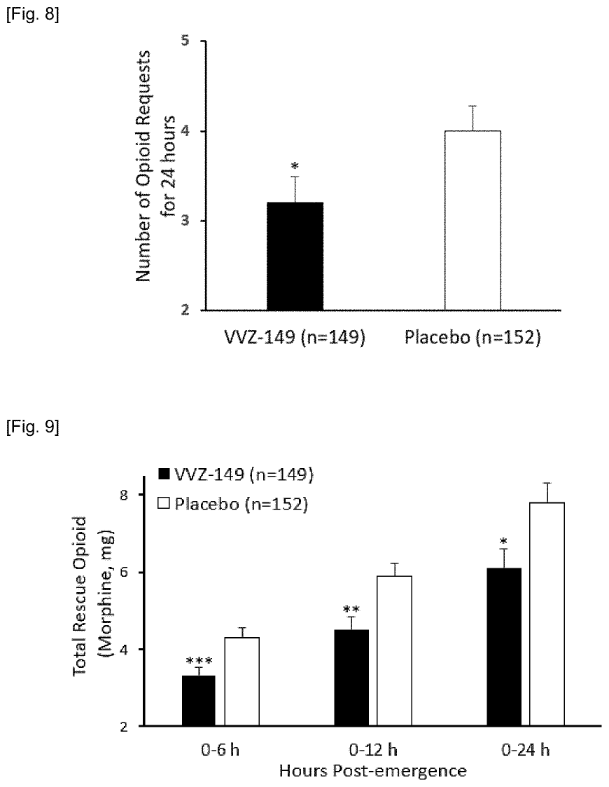

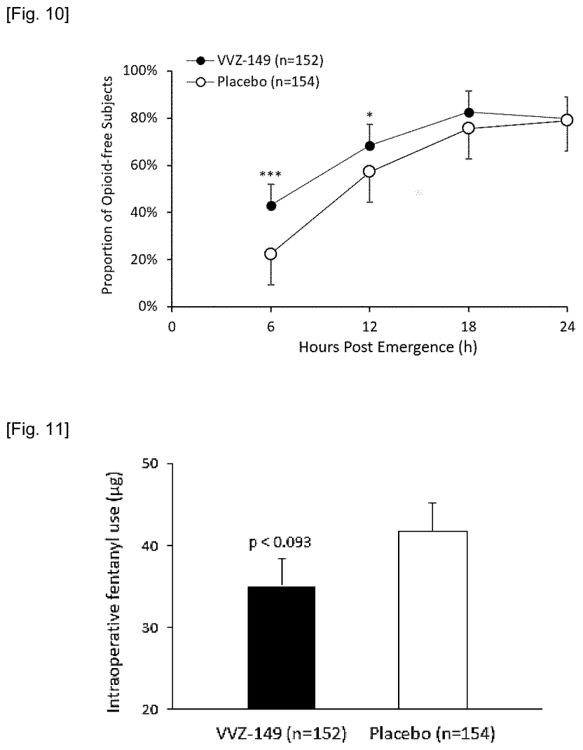

The WZ-149 group showed opioid comparable reduction of postoperative pain with significant less opioid use. The number of requests for rescue opioid and total amount of rescue opioid consumed were significant lower in VVZ-149 group compare to placebo group (FIG. 8 and FIG. 9). Such opioid sparing effect of VVZ-149 was shown in the higher proportion of subjects that are opioid-free during the first 12 hours post-emergence at any given time point (FIG. 10). Considering the strong opioids are mostly required in the early stage of postoperative pain management, the prophylactic treatment with VVZ-149 may facilitate adequate pain relied by the first 12 hours after emergence, while minimizing the need for postoperative opioid. WZ-149 group used less amount of intraoperative fentanyl during surgery than placebo group (FIG. 11).

### Example 3

Sixty subjects aged 18 to 70 for an elective bunionectomy were randomized in a 1:1 ratio to receive either VVZ-149 Injections or a placebo. The WZ-149 group received a 10-hours IV infusion after the nerve block removal in the next morning after surgery at a loading dose of 160 mg for 0.5 h followed by a maintenance dose of 840 mg for 9.5 h, and the placebo group received a placebo with the same dosing regimen. All subjects were provided with IV morphine (2 mg of IV, 15-min lockout interval; not to exceed 4 mg in 1 hours) on demand as rescue dosing to facilitate adequate pain relief. The primary-outcome measure, pain intensity, was evaluated using NRS (0-10) at the scheduled time points through 48 hours post-emergence. Measures of secondary outcomes included the total request and amount of morphine consumed via rescue dosing through 48 hours post-emergence. Subjects were given 2 stopwatches to start at the time of the start of the study drug infusion (T=0) and were instructed to stop the first stopwatch when “perceptible pain relief”, and the second stopwatch when “meaningful pain relief” is felt by the Time 12 hr.

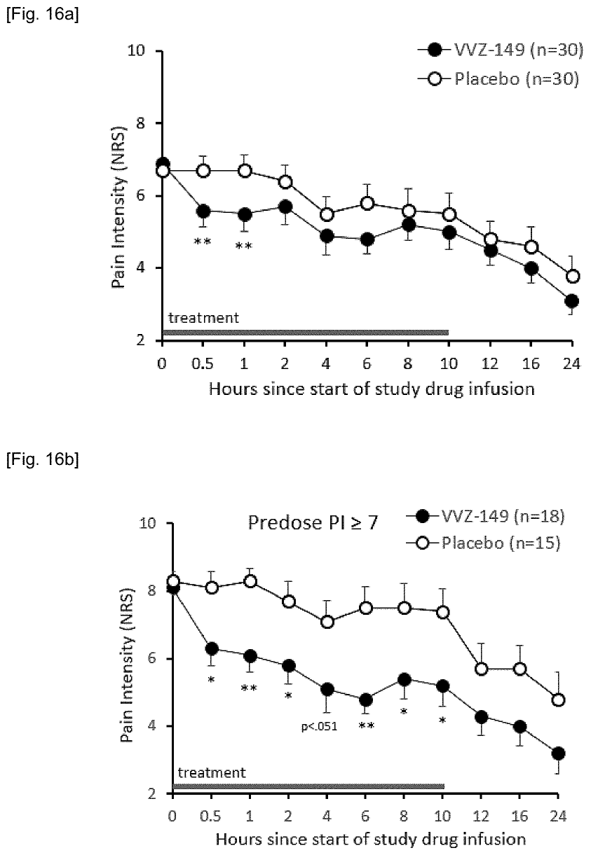

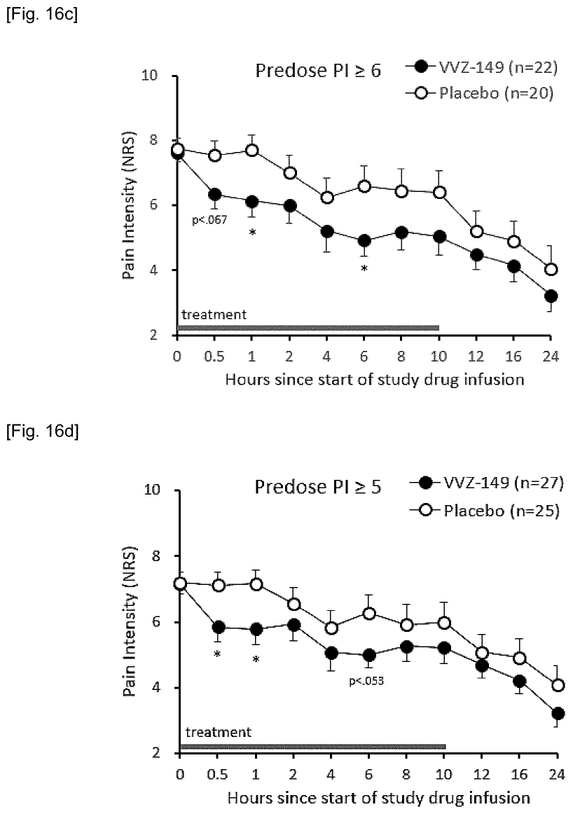

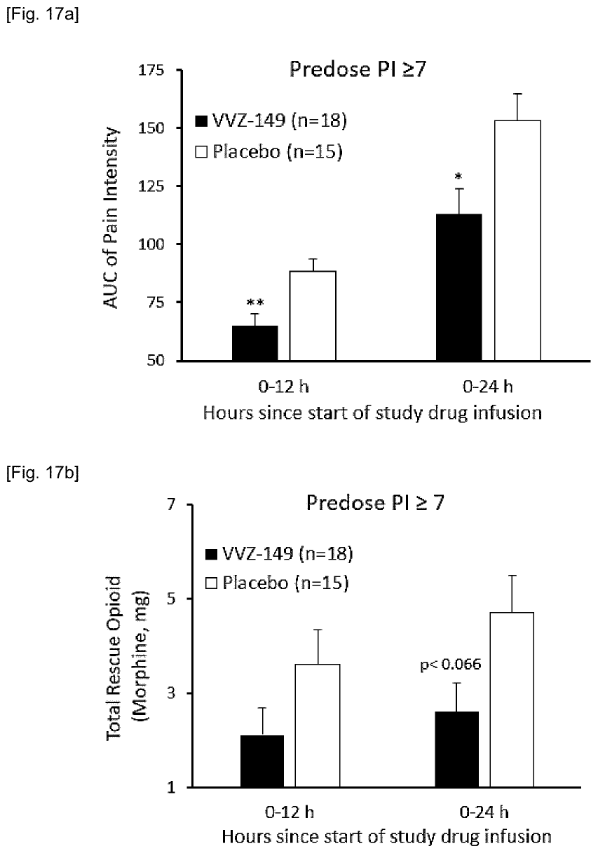

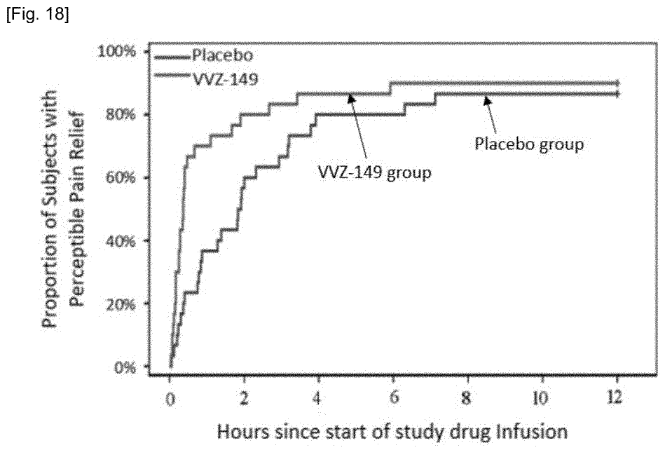

VVZ-149 group showed lower pain intensity with less opioid consumption compared to placebo group (FIG. 16a). More than 50% patients reported perceptible pain relief within 30 minutes after the start of VVZ-149 infusion, indicating the early onset of analgesia with VVZ-149 infusion (FIG. 18). In a subgroup with severe postoperative pain as of predose PI≥7, significant lower pain intensity was reported and significant less morphine was requested and consumed in VVZ-149 subgroup compared to placebo subgroup with severe pain (FIG. 116b, FIG. 17a and FIG. 17b). The results was same in subgroups as of predose PI≥6 or 5 with moderate and severe operative pain (FIG. 16c and FIG. 16d). Effect size of analgesic efficacy gradually enhanced as the subgroup with more severe pain (predose PI≥7, ≥6 or ≥5), despite of the smaller sample size (FIG. 16b, FIG. 16c, and FIG. 16d).

### Example 4

Subjects aged 25-70 years underwent planned laparoscopic or robotic-assisted gastrectomy. Eligible subjects who consented to participate were screened and enrolled in the study and were then randomized in a 1:1 ratio to receive a continuous 10-h intravenous (IV) infusion of VVZ-149 Injections or a placebo for 10 h, starting within 1 h prior to completion of the surgical procedure depending on their randomization assignment (i.e. block randomization).

Subjects aged 25-70 years underwent planned laparoscopic or robotic-assisted gastrectomy. VVZ-149 or placebo was administered intraoperatively within 1 hour prior to the completion of laparoscopic or robot-assisted laparoscopic gastrectomy for a total of 10 hours (loading dose of 1.8 mg/kg for 0.5 hours; maintenance dose of 1.3 mg/kg/hr for 9.5 hours).

In addition to receiving VVZ-149 Injections or the placebo, all subjects were provided with IV patient-controlled analgesia (PCA; fentanyl, 5 μg bolus, 3-min lockout time) on demand to facilitate adequate pain relief after recovering from anesthesia. The time of PCA installation thus corresponded to the initial timepoint of emergence of each subject from anesthesia, referred to in this study as ‘0 h post-emergence’. Additional rescue medication was provided “as needed” if the IV PCA titration did not have adequate pain relief and required by the subject.

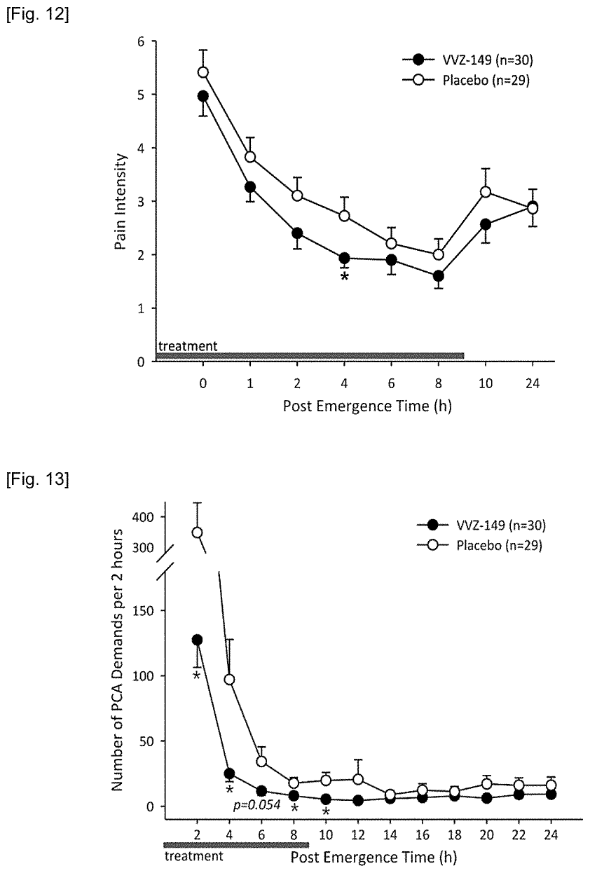

The mean NRS rest in the VVZ-149 group were lower at any given timepoint through 24 hrs post-emergence except at T=24, and statistical significance was achieved at T=4 (p<0.05) (FIG. 12). In alignment with pain relief by VVZ-149, the number of PCA demand by subjects was significantly lessened at T=2 and T=10 (FIG. 13), indicative of a lessened need for an intervention for adequate pain relief.

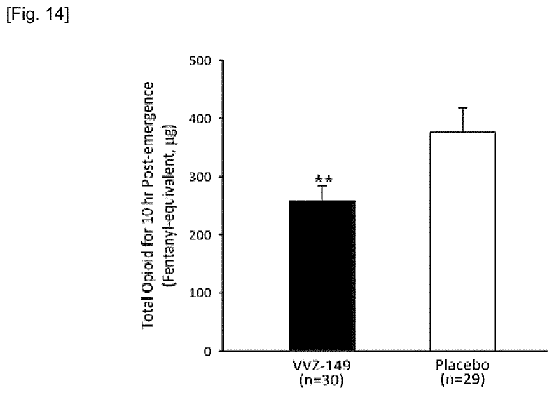

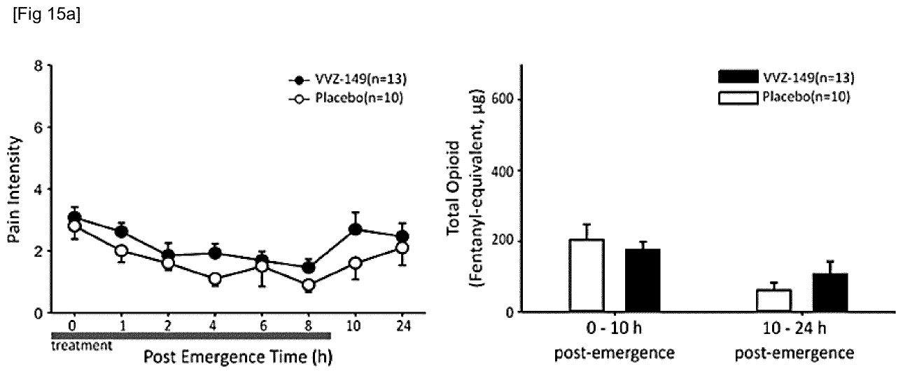

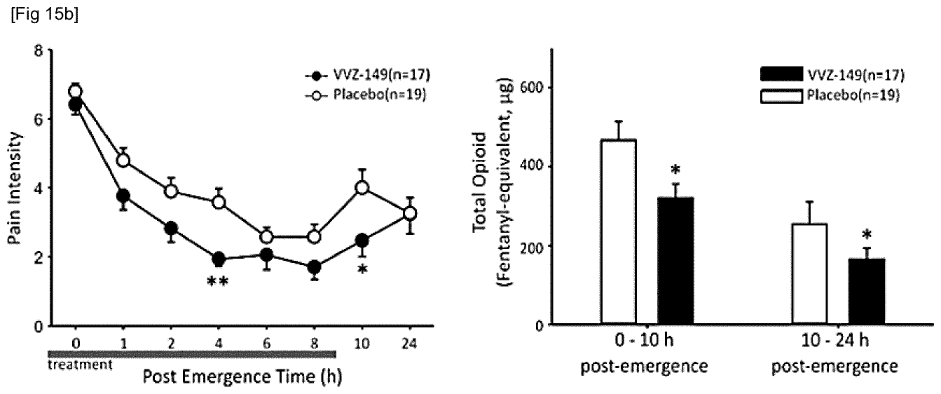

VVZ-149's opioid sparing property was observed as the subjects in the VVZ-149 group consumed significantly less (31.8% opioid sparing) opioid as compared with those in the placebo group for 10 hours post-emergence. (p<0.01) (FIG. 14). Pain intensity at different hours post-emergence and total opioid consumption in rescued and non-rescued groups showed the analgesic effect and the opioid sparing effect of VVZ-149 respectively (FIGS. 15a and 15b). Specifically, in the rescued patient group, PIs in the VVZ-149 group were much lower as compared to the placebo, reaching statistical significance at 4 hr (p<0.01) and 10 hr post-emergence (p<0.05). Respectively, the total amount of opioid consumed reduced by 31.4% and 35% at 0-10 hr and 10-24 hr post-emergence in patients in the VVZ-149 group compared to those receiving placebo (p<0.05).

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method for reducing a need of a postoperative analgesia by a subject underwent operation, comprising administering to the subject an effective amount of a compound of Formula (I): 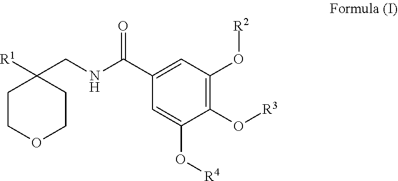
wherein
R¹ is —NR⁵R⁶ or morpholinyl;
R², R³, and R⁴ are each independently selected from the group consisting of alkyl, aryl, and arylalkyl; and
R⁵ and R⁶ are each independently selected from the group consisting of —H, alkyl, and arylalkyl,
or pharmaceutically acceptable salt thereof,
wherein the postoperative analgesia comprises a subject self-administered analgesia, a rescue analgesia, or both;
wherein the subject suffers from postoperative pain and receives an analgesia to treat postoperative pain;
wherein the need of postoperative analgesia comprises one or more of
(i) a frequency of the subject's self-administration,
(ii) an amount of analgesia self-administered by the subject,
(iii) a frequency of rescue request, and
(iv) a total amount of administered postoperative analgesia.

2. The method according to claim 1, wherein the subject is human patient.

3. The method according to claim 1, wherein the postoperative pain is moderate to severe pain.

4. The method according to claim 1, wherein the subject has pain catastrophizing (PCS).

5. The method according to claim 1, wherein the need of a postoperative analgesia is a need during first 72 hours after emergence from anesthesia.

6. The method according to claim 2, wherein the human patient has a capacity of self-administering an analgesia via patient-controlled analgesia (PCA).

7. The method according to claim 1, wherein the compound of Formula (I) is (4-butoxy-N-((4-(dimethylamino)tetrahydro-2H-pyran-4-yl)methyl)-3,5-dimethoxybenzamide) of the following formula: 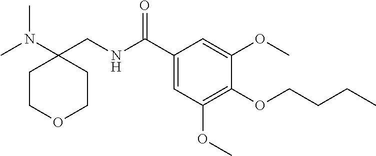 [SMILES: CCCCOc1c(OC)cc(C(=O)NCC2(N(C)C)CCOCC2)cc1OC]
or a pharmaceutically acceptable salt thereof.

8. The method according to claim 1, wherein the compound of Formula (I) or a pharmaceutically acceptable salt thereof is administered continuously via intravenous infusion over about 8 to about 10 hours.

9. The method according to claim 1, wherein the effective amount ranges from about 700 to about 1,400 mg.

10. The method according to claim 1, wherein the administration starts at about 30 minutes prior to anesthesia induction.

11. The method according to claim 1, wherein the administration starts at about 1-2 hours prior to emergence from anesthesia.

12. The method according to claim 1, wherein the administration starts within first 2 hours after emergence from anesthesia.

13. The method according to claim 1, wherein the administration comprises a first dosing of about 50 to about 200 mg for about 0.5-about 1 hour infusion and a second dosing of about 650 to 1,200 mg for about 7 to about 9.5 hours infusion, in this order.

14. The method according to claim 1, wherein the postoperative analgesia is an opioid.

15. The method according to claim 7, wherein the compound of Formula (I) is (4-butoxy-N-((4-(dimethylamino)tetrahydro-2H-pyran-4-yl) methyl)-3,5-dimethoxybenzamide) hydrochloride.

16-34. (canceled)

35. The method according to claim 8, wherein the effective amount ranges from about 700 to about 1,400 mg.

36. The method according to claim 8, wherein the administration starts at about 30 minutes prior to anesthesia induction.

37. The method according claim 8, wherein the administration starts at about 1-2 hours prior to emergence from anesthesia.

38. The method according claim 8, wherein the administration starts within first 2 hours after emergence from anesthesia.

39. The method according to claim 8, wherein the administration comprises a first dosing of about 50 to about 200 mg for about 0.5-about 1 hour infusion and a second dosing of about 650 to 1,200 mg for about 7 to about 9.5 hours infusion, in this order.
